(19) United States
(12) Patent Application Publication
(10) Pub. No.: US 2022/0037034 A1
(43) Pub. Date: Feb. 3, 2022

(54) SYSTEM AND METHOD FOR TRACKING AND TRACING PERSONS WITH SELECTED MEDICAL CONDITIONS

(71) Applicant: AdMoER Inc., Savannah, GA (US)

(72) Inventor: Dragos Stanescu, Savannah, GA (US)

(21) Appl. No.: 17/305,744

(22) Filed: Jul. 14, 2021

Related U.S. Application Data
(60) Provisional application No. 63/068,019, filed on Aug. 20, 2020.
(60) Provisional application No. 63/058,927, filed on Jul. 30, 2020.

(51) Int. Cl. G16H 50/80; G16H 40/67; G16H 50/30; G16H 40/20; G16H 50/70; G06T 11/00; G06F 3/01

(52) U.S. Cl. CPC G16H 50/80; G16H 40/67; G16H 50/30; G16H 10/20; G16H 50/70; G06T 11/001; G06F 3/011; G16H 40/20

(57) ABSTRACT

A system and method for tracking at least healthcare data and/or location data regarding an individual, determining, based on the tracked healthcare data and/or location data, the individual's susceptibility and/or probability of being infected with a contagion and/or infecting others with a contagion, and conveying such determined susceptibility and/or probability to at least the individual.

### PRIORITY CLAIM

This application claims the benefit of and priority to U.S. Provisional Patent Application No. 63/068,019, filed on Aug. 20, 2020, and claims the benefit of and priority to U.S. Provisional Patent Application No. 63/058,927, filed on Jul. 30, 2020, the entire contents of which are each incorporated herein by reference.

### TECHNICAL FIELD

The present disclosure relates to a system and method for tracking and tracing individuals with selected medical conditions. More particularly, various embodiments of the present disclosure provide a system and method for tracking at least healthcare data and/or location data regarding an individual, determining, based on the tracked healthcare data and/or location data, the individual's susceptibility and/or probability of being infected with a contagion and/or infecting others with a contagion, and conveying such determined susceptibility and/or probability to at least the individual.

### BACKGROUND

Global pandemics, such as the outbreak of Coronavirus Disease 2019 (COVID-19), have substantially changed how people interact with each other, such as practicing social distancing in public and/or private spaces. While social distancing helps combat certain global pandemics, such social distancing is being done with a lack of individualized predictive diagnostics (based on disease algorithms and comprehensive risk analysis), with no real time confirmation of risk status or infection status (including identification of those at risk for infection) and with no accurate data available to calculate geographic risk to optimize distribution and dispersion of resources. Couple these limitations with no common global protocol or consolidated data pool (which contributes to confusion and international tensions) and that current monitoring and predictive models are problematic and prone to errors via relying purely on small sample sizes and the associated statistics therefor, and it becomes clear that a need remains for a comprehensive monitoring solution for epidemiological tracing and monitoring which insures individual privacy.

Accordingly, with multiple relatively serious pathogens loose and more nearly certain to become active, a need exists for systems which enable an individual to not only protect their own health but also the health of others and which replace general social distancing with targeted social distancing policies focused on those individuals with elevated risks of infection.

Moreover, with an aging population and a corresponding increase in instances of chronic diseases and illnesses that require long-term and recurrent treatment, a need exists for systems which enable permanent and real-time interaction between an individual and one or more healthcare providers associated with the individual regarding healthcare data associated with that individual to enable the healthcare provider to make real-time decisions regarding a course of treatment for the individual based on the real-time healthcare data of the individual.

### SUMMARY

The present disclosure generally relates to a system and method that monitors those individuals at risk and tracks those who may be contagious with a relatively simple, informative and educational system. In various embodiments, the system monitors individuals by employing one or more healthcare data gathering devices, such as objective healthcare data collected from a wearable device, objective data collected from a mobile phone and/or healthcare data provided by an individual (e.g., answers to a mobile device application prompted questionnaire regarding an individual's health history and any symptoms of any conditions experienced by the individual). Utilizing such healthcare data inputted by and/or collected from the individual, the system of the present disclosure determines one or more healthcare scores to convey to the individual regarding their contagion risk level. In certain embodiments, one such healthcare score pertains to a susceptibility of the individual of being infected with one or more identified contagions based on at least the healthcare data inputted by and/or collected from the individual relative one or more defined comorbidities for those identified contagions, wherein the determined susceptibility of the individual is represented to the individual as a relatively easy to understand numerical score and color code. In certain embodiments, another such healthcare score additionally or alternatively pertains to a probability of an individual being infected with (and correspondingly infecting others with) a contagion based on at least the healthcare data inputted by and/or collected from the individual (and any external input by medical professionals) in accordance with current public health standards, wherein the individual's determined probability of being at risk of infecting others is represented to the individual as a relatively easy to understand numerical score and color code. Accordingly, the present disclosure benefits individuals by providing a positive alternative to isolation strategies (and their accompanying economic devastation) via encouraging best practices for prevention and enabling such individuals to rapidly react to emergency situations which the individual is aware of by changes to their associated numerical score and/or color code.

In addition to providing various benefits to individuals, in certain embodiments, the system and method of the present disclosure provides various public health system benefits via tracking the location of one or more of such individuals (whom have voluntarily enrolled to participate and are tracked anonymously) to enable public health system authorities the ability to monitor different spaces and the healthcare data (and/or healthcare scores) of the anonymous individuals interacting in those different spaces. Such a system thus provides a comprehensive live monitoring system that permits public health authorities the ability to manage resource deployment, economic measures, mitigation efforts, and other activities in emergency situations tied to virulent contagions, such as the COVID-19 crisis. In other words, providing benefits to various health services providers via tracking the location of one or more of individuals (whom have voluntarily enrolled to participate and are tracked anonymously) to enable such health services providers the opportunity to build predictions associated with the flow of individuals (whom may or may not be infected with a contagion) enables optimal allocation of logistical resources, medical resources and/or human resources when needed most.

In certain embodiments, in addition to aiding public health system authorities, the system and method utilizes part or all of such tracked locations and healthcare data (and/or healthcare scores) of the anonymous individuals interacting in different spaces, such as various businesses in usually crowded locations (e.g., hotels, restaurants and bars, office buildings, industrial facilities, event venues, airports and train stations), to provide the operator of such spaces with various information regarding the infection risk of anonymous individuals located within or approaching a specific space to enable the operator of such a space the opportunity to modify one or more policies to create a relatively safer environment for such anonymous individuals located within or approaching that specific space.

In certain embodiments, the system and method of the present disclosure additionally or alternatively tracks certain healthcare data, such as signs and symptoms associated with an individual, and/or location data regarding an individual with a known chronic disease condition (e.g., heart diseases, nutrition and metabolic diseases, oncologic conditions, pulmonary diseases). In these embodiments, the system and method determines, based on the tracked healthcare data and/or the location data, information associated with the individual and transfers, in real time, the determined information to a healthcare provider and/or healthcare facility associated with the individual to aid the healthcare provider and/or healthcare facility in determining a need for medical intervention. That is, the real time monitoring of an individual with a known chronic disease condition that requires long-term and recurrent treatment coupled with the communication, to a healthcare provider and/or healthcare facility associated with that individual, of pertinent monitored information associated with that individual, such as any alarming risk levels currently associated with that individual, provides an improved disease and therapeutic management system with a positive impact on the individual's monitored health condition. In certain such embodiments, the system and method utilize disease specific built algorithms to facilitate the real time transmission of risk levels and/or alarms associated with an individual to enable the healthcare provider and/or healthcare facility in making a proper, real-time assessment of therapeutic progress and/or intervention as needed.

These and other embodiments, and various permutations and aspects, will become apparent and be more fully understood from the following detailed description and drawings, which set forth illustrative embodiments that are indicative of the various ways in which the principles of the invention may be employed.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A, 1B, 1C, 1D, 1E, 1F, 1G, 1H, 1I, 1J, and 1K are flow charts of example processes for operating the system of various embodiments of the present disclosure.

FIGS. 2A, 2B, 2C, 2D, 2E, 2F and 2G are example graphical user interfaces displayed on a mobile device in connection with the system of various embodiments of the present disclosure.

### DETAILED DESCRIPTION

The description that follows describes and exemplifies one or more particular embodiments of the invention in accordance with its principles. This description is not provided to limit the invention to the embodiments described herein, but rather to explain and teach the principles of the invention in such a way to enable one of ordinary skill in the art to understand these principles and, with that understanding, be able to apply them to practice not only the embodiments described herein, but also other embodiments that may come to mind in accordance with these principles. The scope of the invention is intended to cover all such embodiments that may fall within the scope of the appended claims, either literally or under the doctrine of equivalents.

With respect to the exemplary systems, components and architecture described and illustrated herein, it should also be understood that the embodiments may be embodied by, or employed in, numerous configurations and components, including one or more systems, hardware, software, or firmware configurations or components, or any combination thereof, as understood by one of ordinary skill in the art. Accordingly, while the disclosure explains exemplary systems including components for one or more of the embodiments contemplated herein, it should be understood that with respect to each embodiment, one or more components may not be present or necessary in the system.

It should also be noted that the disclosures made in this specification are in accordance with the principles of the embodiments(s), which are intended to be disclosed or interpreted to their broadest extent under the patent laws of the United States and other countries, and while such disclosure may describe or otherwise cover subject matter that may be regulated by other existing laws or regulations in the United States and other countries, including, without limitation, the Health Insurance Portability and Accountability Act (HIPAA), nothing in this disclosure is intended to suggest or imply noncompliance with any such law or regulation by the assignee.

In various embodiments, the present disclosure is directed to a system which assesses the risks of an individual of both being contagious with and becoming infected by a pathogen, even pathogens not yet specifically identified. In these embodiments, for certain individuals, such as relatively healthy individuals whom have accessed the system via a mobile device application, the system assigns a risk of that individual carrying a pathogen based on system identified exposure, medical information provided by the user (via one or more healthcare data gathering devices and/or data inputted into a mobile device application regarding any infection symptoms and/or healthcare history) and any medical data provided by public health and medical professionals (via data obtained by a mobile application, a web application running on a personal computer and/or a web browser). Having assigned a risk of that individual carrying a pathogen, the system utilizes the mobile device application, a web application and/or any suitable display device to convey to the individual their infection risk level via a numerical score and color code, wherein different numerical scores and color codes are associated with different infection risk levels.

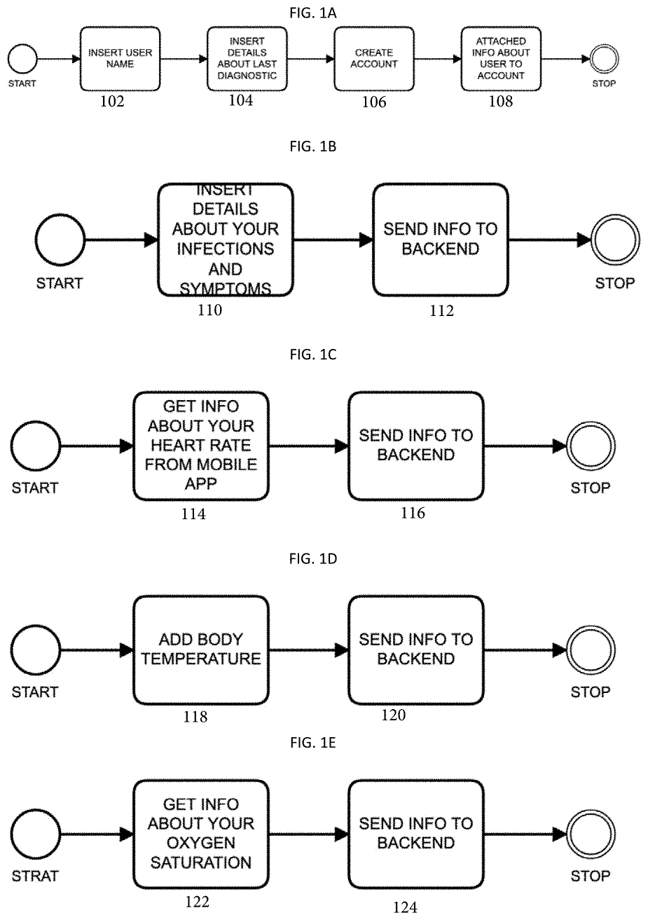

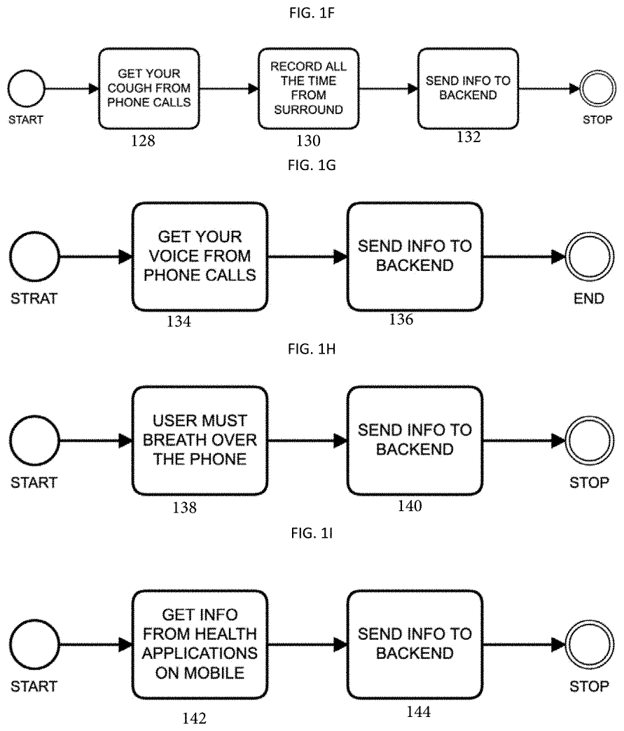

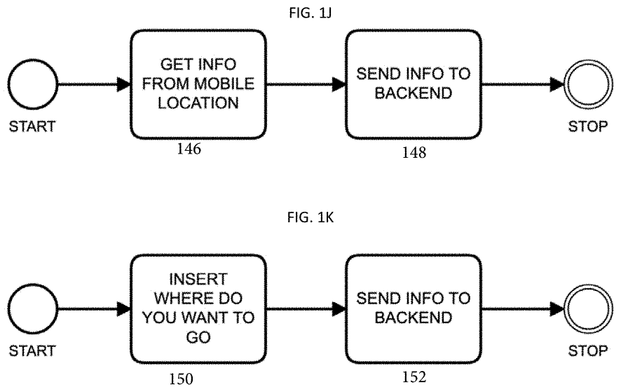

More specifically, in certain embodiments, to create an account and enable the system to track and trace one or more elements of an individual's health and/or location, as seen in FIG. 1A, following the system obtaining a user name for a new user (as seen in block 102) as well as diagnostic details about the new user (as seen in block 104), the system creates an account for the new user (as seen in block 106) and attaches information about the new user to the created account (as seen in block 108). It should be appreciated that upon an individual opting to participate in the tracking and tracing system of the present disclosure via a voluntary submission by the individual, the system assigns the individual a unique character ID (i.e., an identity code such as a six character alpha numeric identifier) wherein only that character ID is used to anonymously identify the individual. For example, the system only uses the character ID associated with an individual when a remote query is run by any third-parties, such as any public health institutions, thereby eliminating the requirement to provide any personal identifiable information of any individuals when responding to such remote queries. In these embodiments, the system assigns to each individual end-user a unique ID generated and recognized by an employed artificial intelligence (“AI”) security solution, wherein the system does not share any private data regarding the end-user. Specifically, the employed AI security solution continuously monitors the data flows from end-users to core neural nets and application programming interfaces of the disclosed system and thoroughly checks all data points to guard against any potential privacy concerns. Moreover, the system stores the specificity of all data points on the end-user's device wherein the system resolves various outputs that require private data directly on the end-user's device and only exports towards the core neural net said outputs for processing thereby protecting end-users anonymity not only to third parties (e.g., public health authorities and/or private organizations) but the administrators of the system itself

In certain embodiments, in addition to assigning a unique character ID and only in certain situations (e.g., at the request of an authorized government institution or the request of a user's employer following the express approval of the user which is only disregarded in case of an issued legal mandate), the system retains an image of user, such as taken with a device's camera or otherwise transmitted from the device. In one such embodiment, if the individual assigned the unique character ID is subsequently associated with an assigned risk/warning code, the system utilizes the image of the individual for health care status verification purposes. In another such embodiment wherein the system retains (in a separate database) or otherwise has access to an image of a government issued identification, if the individual assigned the unique character ID is subsequently associated with an assigned risk/warning code, then responsive to a query by a public health department, the system utilizes the image of the government issued identification of the individual for health care status verification purposes.

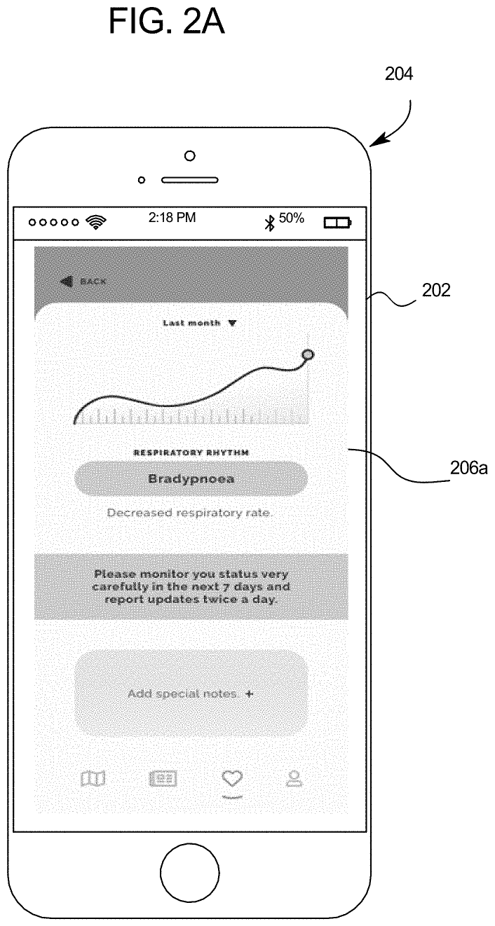

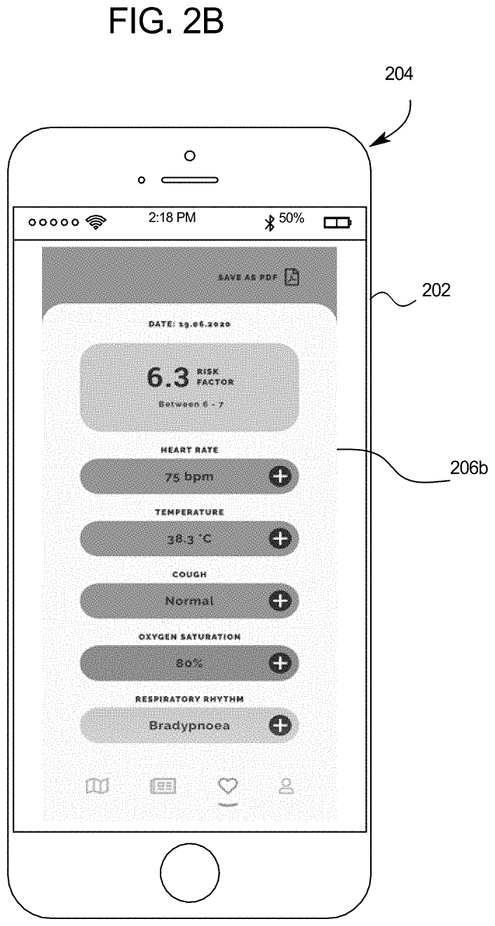

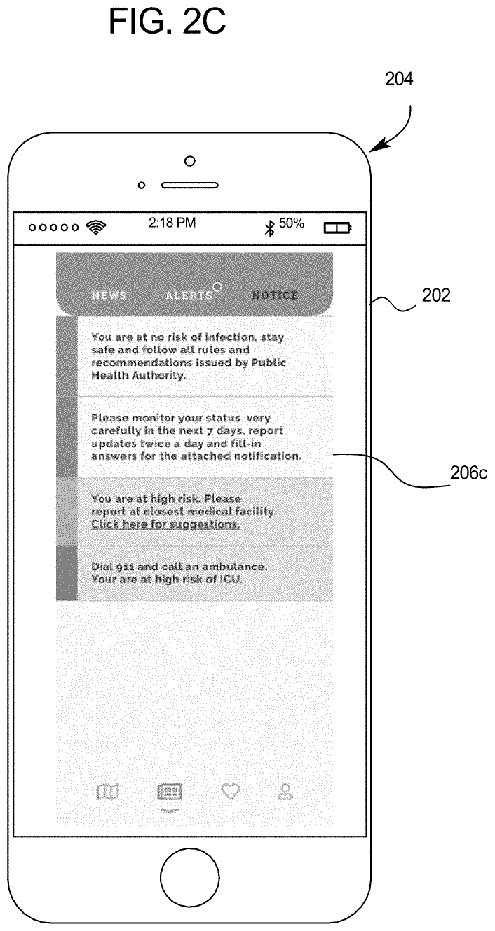

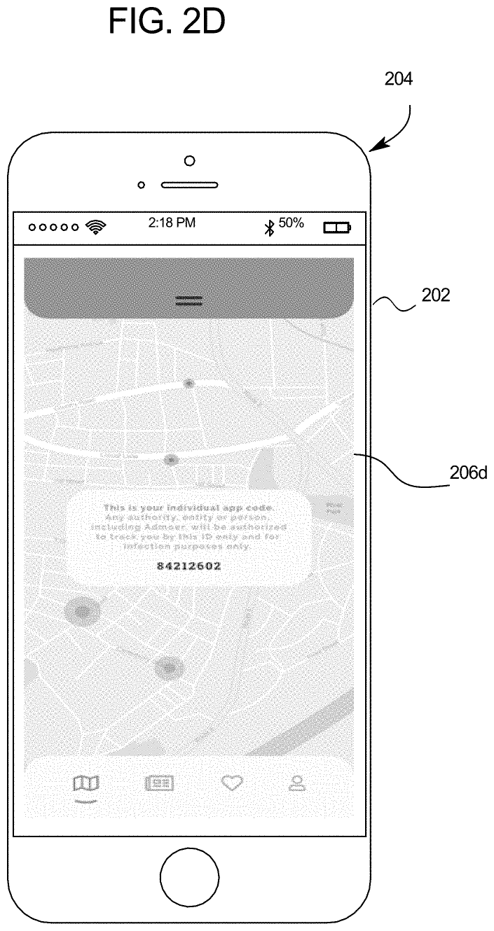

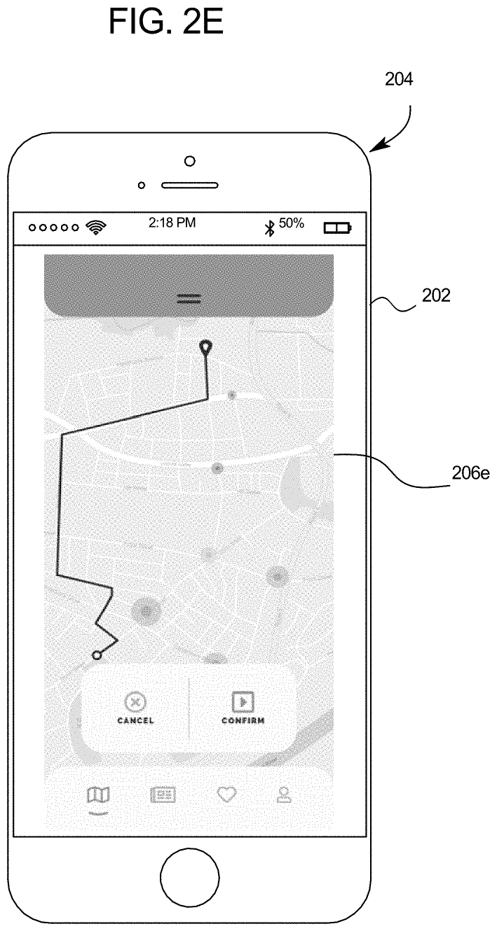

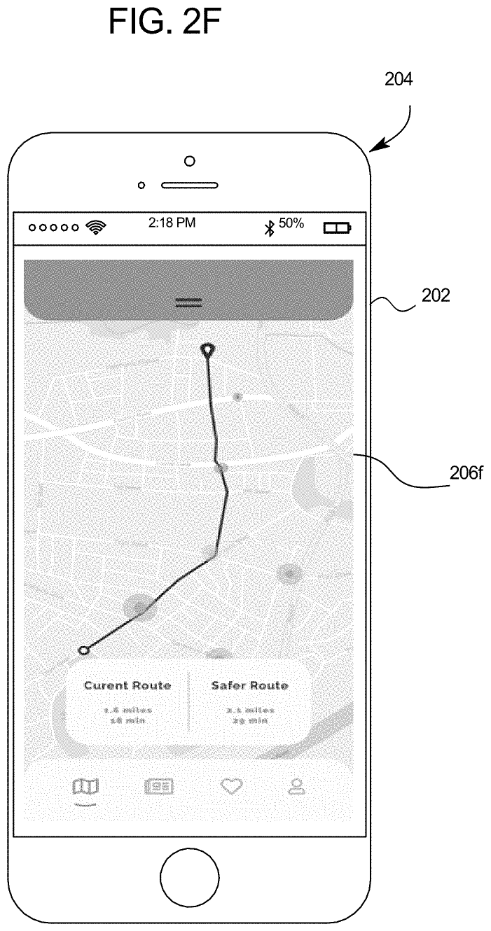

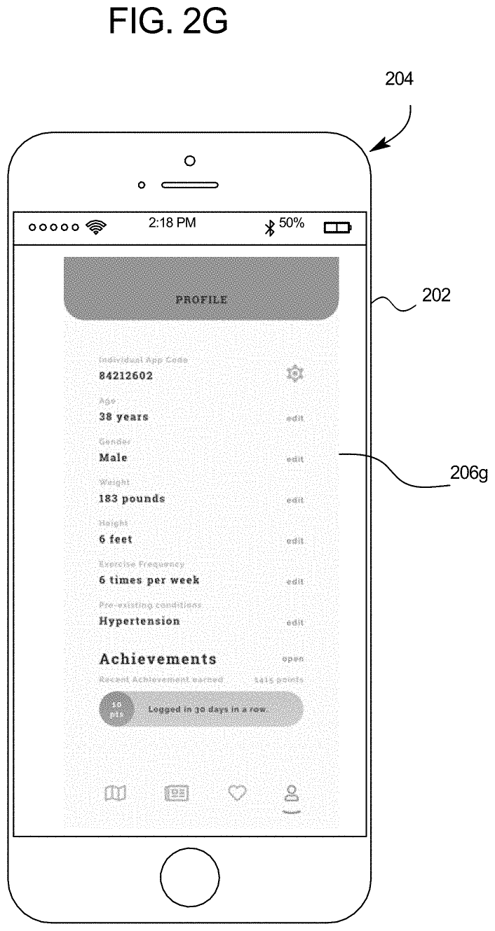

In various embodiments, the system of the present disclosure tracks various healthcare data associated with an individual including, but not limited to, user-inputted general/health history data (e.g., age, sex, height, weight, frequency of exercise, pre-existing conditions and/or critical but transitory conditions, such as cancers and/or physical injuries), user-inputted subjective clinical data (e.g., signs and symptoms of temperature, chest pains, sputa, headaches, anosmia, sneezing, stuffed nose, and/or rhinorrhea), user-inputted chronic disease data (e.g., cardiovascular, respiratory, auto-immune, oncologic, bacterial infection, and/or viral infection), and/or non-user-inputted objective clinical data (e.g., heart rate, oxygen saturation, respiratory rhythm, specific vocal patterns, dry cough, dyspnea and/or sleep patterns) obtained via one or more sensors in communication with the system. In one such embodiment, the system utilizes a mobile device application (or a website interface) to display to the individual one or more current and/or historical components of the user-inputted general/health history data, the user-inputted subjective clinical data, the user-inputted chronic disease data, and/or the non-user-inputted objective clinical data. For example, as seen in FIGS. 2A and 2B, the mobile device application 202 of a mobile device 204 displays to the user various healthcare data 206a and 206b obtained via one or more sensors in communication with the system as well as a determined risk factor (as seen in FIG. 2B and described below) and one or more messages associated with the determined risk factor (as seen in FIG. 2A and also described below).

In certain embodiments and following an individual specifically agreeing to a disclosure of any information associated with the individual to a medical care professional associated with the individual, the system of the present disclosure additionally or alternatively tracks an individual's associated medical care professionals, including, but not limited to, their primary physician, any specialists and a preferred medical center. In certain embodiments, the system enables the individual's associated medical care professionals to input one or more of the user-inputted general/health history data, the user-inputted subjective clinical data, and/or the user-inputted chronic disease data. In certain embodiments, the system of the present disclosure additionally or alternatively tracks an individual's vaccination history (which requires confirmation by a medical professional associated with the individual).

Moreover, to function as a warning system to multiple users (as described below), in certain embodiments, the system additionally or alternatively tracks an individual's location (with the individual's anonymity being preserved) and corresponding exposure to public health defined critical infectious organisms (e.g., COVID-19, SARS, TB) which can be entered by public health departments or medical professional, wherein such location data factors into determining an appropriate risk code as described herein.

It should be appreciated that since the system tracks various healthcare data, the system encrypts all personal identifying information in such a way that only the individual has access to their data. That is, while in certain embodiments, the system enables one or more servers of the system to interface with one or more servers of a public health department system to run a remote query of the status of one or more individuals (for health care status verification purposes), any provided information is encrypted such that the identity of any individual identified in the query is unknown. In other words, once assigned an anonymous identity code by the system, only that anonymous identity code is tracked by the system and only that anonymous identity code is used by any authority, entity or person to identify the individual. As such, the system maintains anonymity of the application end-users, wherein a full anonymity enrollment process is carried out including upon enrolling in the program provided by the present disclosure, the new user creates a password for their account and the system assigns an anonymous identity code to the created account. In these embodiments, the system maintains that each unique identity code is paired with the serial number of the mobile device executing the application such that the identity of the end-user cannot be traced back to that end-user even by administrators of the system.

It should be appreciated that while the system provides that the end-users of the system remain anonymous from other end-users of the system and from the administrators of the system, in certain limited instances, the system enables certain healthcare providers (e.g., doctors and/or hospitals) to match an end-user of the system with the end-user's assigned anonymous identity code in accordance with applicable doctor-patient confidentiality. In these embodiments, as an additional safeguard against inadvertently identifying an end-user of the system, the system requires a healthcare provider to introduce additional verification data, such as test results, hospital release forms and/or proof of vaccinations, in association with the unique identity code assigned to the anonymous user (and not in association with any identity of any end-user of the system). In such embodiments, after the system verifies that the additional verification data provided by the healthcare provider matches the data stored by the system in association with the end-user will the system enable the healthcare provider to match the identification of an individual user with the unique identity code assigned to that user. Accordingly, the employment of anonymous identity codes coupled with the obligation of healthcare providers to not disclose an individual's identity in accordance with physician-patient privilege results in the system maintaining the anonymity of each participating individual.

In addition to maintaining anonymity of the participating individuals via the use of anonymous identity codes, in certain embodiments, the system of the present disclosure employs a medical predictive algorithm which enables for processing and determining infection risk levels based on direct or indirect reading of healthcare data via one or more of a mobile phone, a wearable device (i.e., a wearable device including one or more sensors configured to obtain clinical healthcare data of the wearer), healthcare data entered by the individual (e.g., answers to an application user questionnaire of infection symptoms) and/or healthcare data entered by the individual's authorized healthcare professional (e.g., vaccination history). In these embodiments, the system compares such sensed healthcare data and/or inputted healthcare data to information associated with existing symptoms in one or more databases maintained by the system or otherwise in communication with one or more servers of the system.

In certain embodiments, the system employs continuous (or periodic) synchronization with the healthcare data entered or otherwise communicated via the application by the individual and/or their authorized healthcare professional (whom may communicate with the system via a healthcare professional application, via a web application running on a personal computer and/or via a web browser), wherein the system communicates such obtained healthcare data to one or more servers for further analysis. For example, as seen in FIG. 1B, the system obtains details about an individual's infections and symptoms (as seen in block 110) and sends such obtained information to one or more servers for storage and/or analysis (as seen in block 112).

In certain embodiments, the system additionally or alternatively employs continuous (or periodic) synchronization with the individual's mobile phone and/or wearables sensors to collect objective clinical healthcare data (e.g., heart-rate, oxygen saturation levels, respiratory rhythm, temperature, specific vocal patterns, dry cough, dyspnea and sleep patterns), wherein the system communicates such collected objective clinical healthcare data to one or more servers for further analysis. In these embodiments, as described above, any communicated collective objective clinical healthcare data (as well as any communicated user-inputted general/health history data, any communicated user-inputted subjective clinical data, and/or any communicated user-inputted chronic disease data) is encrypted and depersonalized (i.e., associated with the individual's unique character ID only such that the identity of the individual is anonymous and separate from the individual's communicated healthcare data).

In various examples, to detect heart-rate, body temperature and/or oxygen saturation, the system obtains data from one or more wearable devices equipped with one or more sensors to determine heart-rate (as seen in block 114 of FIG. 1C), body temperature (as seen in block 118 of FIG. 1D) and/or oxygen saturation (as seen in block 122 of FIG. 1E) and sends such obtained information to one or more servers for storage and/or analysis (as seen in blocks 116, 120 and 124 of FIGS. 1C, 1D, and 1E, respectively). In another example, as seen in FIG. 1F, to detect cough peak flow data, the system obtains cough data from one or more phone calls conducted with the individual's mobile phone (as seen in block 128), records all the time from the surroundings (as seen in block 130) and sends such obtained information to one or more servers for storage and/or analysis (as seen in block 132). In another example, as seen in FIG. 1G, to detect the individual's specific vocal pattern data, the system obtains vocal pattern data from one or more phone calls conducted with the individual's mobile phone (as seen in block 134) and sends such obtained information to one or more servers for storage and/or analysis (as seen in block 136). In certain embodiments, using data associated with the individual's voice obtained via one or more recorded and/or live phone calls made with the individual's mobile phone, the system collects objective clinical healthcare data regarding the individual's specific vocal pattern data (and additionally or alternatively the individual's cough peak flow and/or the individual's respiratory flow). In certain such embodiments, the system transforms any voice frequency recording into one or more “images” and detects any abnormal patterns which are then run through or otherwise compared against one or more algorithm definitions developed by medical and healthcare experts to map the detected abnormalities to their respective subjective and/or objective clinical signs and/or symptoms (to provide the individual immediate feedback via the application including any recommended courses of action).

In other embodiments, to detect cough peak flow data, specific vocal pattern data and/or respiratory flow data, the system utilizes the individual's mobile phone to collect audio data associated with the individual (i.e., the individual breaths over the mobile phone). For example, as seen in FIG. 1H, the system obtains the individual's respiratory flow data via the user breathing over the mobile phone (as seen in block 138), such as a predetermined distance from the microphone of the mobile phone, wherein the system sends such obtained information to one or more servers for storage and/or analysis (as seen in block 140). In these embodiments, using data associated with the recorded and/or live sounds captured by the individual's mobile phone, the system collects objective clinical healthcare data regarding the individual's respiratory flow (and additionally or alternatively the individual's cough peak flow and/or the individual's specific vocal pattern data). In certain such embodiments, the system transforms any audio data recordings into one or more “images” and detects any abnormal patterns which are then run through or otherwise compared against one or more algorithm definitions developed by medical and healthcare experts to map the detected abnormalities to their respective subjective and/or objective clinical signs and/or symptoms (to provide the individual immediate feedback via the application including any recommended courses of action).

In another example, in addition to or alternative from collecting objective clinical healthcare data from one or more wearable devices, the system obtains such objective clinical healthcare data (and/or other healthcare data disclosed herein) from one or more healthcare applications being executed on the individual's mobile phone (as seen in block 142 of FIG. 1I). In this example, the healthcare application being executed on the individual's mobile phone may or may not be obtaining healthcare data about the individual from the mobile phone and/or any wearable devices in communication with the mobile phone. In this example, following obtaining such information, the system sends such obtained information to one or more servers for storage and/or analysis (as seen in block 144).

In addition to collecting subjective healthcare data of an individual and/or objective clinical healthcare data of the individual, the system continuously (or periodically) compares the data reading with symptoms and parameters existing in context relevant databases. Such comparisons enable the system to detect when the participating individual's healthcare data indicates the presence or potential presence of one or more contagions. In certain embodiments, to analyze the individual's healthcare data, the system employs one or more medical predictive algorithms that are based on a decision tree utilizing existing Centers for Disease Control and Prevention (“CDC”) and/or World Health Organization (“WHO”) protocols (e.g., for COVID-19; for seasonal flu and respiratory viruses; for comorbidities such as diabetes, obesity, heart and circulatory system diseases, oncological diseases, respiratory system diseases) correlated between each other. Specifically, the predictive algorithm and objective clinical data readings are bi-directionally modeled using decision tree learning AI, wherein, when appropriate, the AI will provide the user with an additional in-application questionnaire to gather subjective clinical data, signs and symptoms (e.g., temperature, dry cough, dyspnea, chest pains, fatigue, sputa, anosmia, headaches, sneezing, stuffed nose and/or rhinorrhea) in connection with specific comorbidities symptoms (e.g., hypertension, diabetes, oncological conditions, and/or obesity) to include the findings in the decision tree and reiterate the decision tree to further refine the risk assessment and enhance the risk assessment outcome. Put differently, the application employed by the system of the present disclosure monitors, for each participating individual, objective signs and symptoms (collected via one or more wearable devices, via one or more mobile phones and/or via direct input by the individual or a third party associated with the individual (e.g., a healthcare provider), wherein the AI algorithm creates and maintains a comprehensive risk assessment for all participating individuals such that public health departments may access to the full suite of features for such anonymous individuals in the event they need to track and trace a public health issue.

In various embodiments, utilizing at least one of the objective healthcare data obtained via one or more sensors associated with an individual's mobile phone and/or wearable devices (e.g., smart watches, wireless communication enabled sensors, such as arterial-blood gas sensors, temperature sensors, heart rate sensors, EKG sensors, sleep pattern sensors) and the data provided by the individual, such as via answers to questions prompted by an application, the system determines an individual's susceptibility (i.e., fragility) of being infected with a contagion based on the employed CDC/WHO defined comorbidities for identified contagions, wherein the fragility is represented as a relatively easy to understand numerical score and color code. That is, the system assigns risk codes for infection probability via AI algorithm wherein, as described below, the risk code is updateable by subsequent contacts with/avoidance of infected or suspected infected individuals and individuals are alerted upon the modification of their associated risk code.

Moreover, in various embodiments, utilizing at least one of the objective healthcare data obtained via one or more sensors associated with an individual's mobile phone and/or wearable devices (e.g., smart watches, wireless communication enabled sensors, such as arterial-blood gas sensors, temperature sensors, heart rate sensors, EKG sensors, sleep pattern sensors) and the data provided by the individual via answers to questions prompted by an application, the system determines an individual's probability of being infected (and therefor infecting others) with a contagion, wherein the determination is based on data obtained by the application (and from any external input by medical professionals) in accordance with the employed CDC/WHO standards. In these embodiments, the system represents the possibility of being at risk of infecting others as a relatively easy to understand numerical score and color code. That is, the system assigns warning codes for the probability of infecting others via the AI algorithm wherein, as described below, the warning code is updateable by subsequent contacts with/avoidance of infected or suspected infected individuals and individuals are alerted upon the modification of their associated warning code.

In various embodiments, in determining an individual's susceptibility of being infected with a contagion (wherein different numerical scores and/or color codes are associated with different susceptibilities of being infected) and/or in determining an individual's probability of being infected and therefor infecting others with a contagion (wherein different numerical scores and/or color codes are associated with different probabilities of being infected), the system quantifies different signs and symptoms of different ailments by assigning different numerical values to the presence or absence of such signs and symptoms. For example, for the symptom of a loss of smell, the system assigns a first value to an individual whom suffers from a loss of smell (as determined subjectively by the individual or objectively via one or more devices in communication with the system) and assigns a second, different value to an individual whom suffers from no loss of smell (as determined subjectively by the individual or objectively via one or more devices in communication with the system).

In addition to quantifying different symptoms with different ailments, in these embodiments, aware that certain comorbidities factor into an individual's susceptibility of being infected with a contagion and/or an individual's probability of being infected, the system additionally quantifies different comorbidities by assigning different numerical values to the presence or absence of such comorbidities. For example, the system assigns a first value to an individual whom suffers Type I diabetes and assigns a second, different value to an individual whom suffers Type II diabetes.

Moreover, in these embodiments, aware that an individual's age factors into that individual's susceptibility of being infected with a contagion and/or that individual's probability of being infected, the system additionally quantifies different ages by assigning different numerical values to such ages. For example, the system assigns a first value to an individual aged sixty to seventy years old and a second, different value to an individual over seventy years old.

In various embodiments, after assigning values to different symptoms (or the lack of such symptoms) exhibited by an individual and factoring in any comorbidities of the individual and an age of the individual, the system generates a score wherein the score corresponds to a state of the individual (e.g., healthy, in need of self-monitoring, in need of testing, emergency, and emergency with a high risk of need of intensive care unit) and recommended next steps. For example, a score (which the system color codes for relative each of use) corresponding to the individual having a relatively high probability of being infected is associated with the recommended next steps of placing an emergency call to obtain assistance. On the other hand, a score (which the system color codes for relative each of use) corresponding to the individual having a relatively low susceptibility of being infected with a contagion and/or a relatively low probability of being infected is associated with the recommended next steps of continuing to self-monitor for any of the signs and symptoms accounted for by the system.

It should be appreciated that in various embodiments, if an active risk of infection is discovered in an individual, an appropriate code will be assigned, such as a warning code that the individual is infected with a contagion. In these embodiments, while the code may be assigned by the individual or a health care professional, the individual cannot clear the code (thereby preventing individuals from inadvertently designated themselves as relatively low risk of infecting others when they remain relatively high risk of infecting others). Rather, following one or more healthcare professionals (or systems associated with such healthcare professionals) introducing healthcare data, such as test results, hospital discharge documents and/or vaccination status, to the system, the medically predictive algorithm executed by the AI of the application of the present disclosure accounts for such healthcare professional introduced healthcare data and causes a change of the risk level of the individual, such as clearing the warning code associated with an individual being infected with a contagion and reassigning a different color and/or numerical indicator associated with that individual's state of health. As such, while individuals and/or healthcare professionals periodically introduce different data to the system (based on direct or indirect reading of healthcare data via one or more of a mobile phone and/or a wearable device, based on healthcare data entered by the individual and/or based on healthcare data entered by the individual's authorized healthcare professional), the system of the present disclosure accounts for such different data at different points in time to determine the level of risk of that individual (at the current point in time) and the corresponding numerical score and/or color code to assign to that individual.

It should be further appreciated that in certain embodiments, the system utilizes the application to provide messaging to individuals based on their determined risk or warning code (e.g., messages to seek medical care) and/or any changes or alerts issued by public health departments. For example, as seen in FIG. 2C, the mobile device application 202 of a mobile device 204 displays different color coded messages 206c (displayed at different points in time but displayed together for illustration purposes) ranging from “You are at no risk of infection” to “You are advised to dial 911 to check if you need to call an ambulance” (not shown) to “Dial 911 and call an ambulance” to advise an individual on next steps to take after the system has determined a susceptibility of being infected with a contagion and/or a probability of being infected.

In various embodiments, in addition to conveying information to the individual user of the application using relatively easy to understand numerical scores and color codes, the numerical scores and color codes include a healthcare status verification component wherein the numerical scores and/or color codes ensure that those that enter a building or venue do not pose a risk (relative to the contagion in question). That is, in certain embodiments, the system of the present disclosure utilizes an application, such as a mobile device application, to display dynamic health/risk statuses which may be used to verify a healthcare status of an individual resulting in the permission of that individual to entry a building or other environment wishing to reduce the risk to all occupants. In one such embodiment, the healthcare status verification component of the system includes an individual using an application being executed on their device, such as their mobile phone, to display the determined numerical score and color code to convey to others that the individual does not pose a risk (relative to the contagion in question). In certain embodiments, the application being executed on a device additionally display a picture of the individual to verify the person with the device is the same person associated with the determined numerical score and color code. In another such embodiment, the healthcare status verification component of the system includes one or more servers of the system communicating information associated with an individual (e.g., the determined numerical score and color code) to one or more servers of a third party to convey to the third party system that the individual does not pose a risk (relative to the contagion in question) and should be granted access to a building or venue maintained by the third party.

In addition to assessing the risks of an individual, in certain embodiments, the system functions as a warning system to users regarding the risk of others potentially being infected. For example, as seen in block 146 of FIG. 1J, the system obtains mobile location data from an individual (via the individual's mobile phone and/or location-enabled wearable device(s)) and, as indicated in block 148 of FIG. 1J, sends such obtained location information to one or more servers for storage and/or analysis. In these embodiments, for certain individuals, such as individuals with documented pre-existing conditions, the system assigns a risk of that individual becoming infected and further utilizes location data of that individual (and other individuals in the area) to notify others in the area that an individual in the area is medically fragile and requires additional social distancing to assist in their personal safety. Similarly, if the system determines that an individual has been exposed or has tested positive for a pathogen, the system not only utilizes the mobile device application to convey to the individual their increased infection risk level via a modified numerical score and color code, the system also utilizes location data of that individual (and other individuals in the area) to notify others in the area that a possibly contagious individual is in the area so they can take appropriate social distancing precautions.

In addition to assessing the risks of an individual, in certain embodiments, the system functions as a warning system to users regarding the risk of other users potentially being infected. For example, the system obtains location data from an individual (such as via one or more of the individual's mobile phone or location-enabled wearable device) and communicates such obtained location data to one or more servers for storage and/or analysis. In these embodiments, for certain individuals, such as vaccinated individuals, the system assigns a distinct icon, such as symbol of a shield together with a displayed risk of that individual becoming infected, and further utilizes location data of that individual (and/or other individuals in the area) to notify others in the area that an individual in the area is vaccinated and poses a relatively lower risk of infection.

In certain embodiments, the system utilizes the location data from multiple users to advise one or more users on directions to one or more locations. For example, as seen in block 150 of FIG. 1K, the system obtains destination data from an individual (such as via the individual's mobile phone) and, as seen in block 152 of FIG. 1K, sends such obtained destination information to one or more servers for storage and/or analysis. In these embodiments, the mobile device application includes a mapping/direction function wherein upon a user selecting a destination location, the application utilizes the location of various anonymous users and the numerical scores of such anonymous users to determine directions to the destination location that provides the relatively safest route (as it pertains to the user avoiding areas where certain other possibly contagious individuals are currently located). For example, as seen in FIGS. 2D to 2F, following an individual (whom the mobile device application 202 of the mobile device 204 only identifies by their displayed unique character ID 206d of 84212602 as seen in FIG. 2D) making one or more inputs in the mapping/direction function to indicate that they want directions from their home to a destination and following the user selecting a first route (i.e., the displayed route 206e in FIG. 2E) to the destination, the system determines that the presence of other possibly contagious individuals tracked by the system whom are now located along the first route should be minimized or avoided. In this example, to account for these other individuals, the mobile device application 202 of the mobile device 204 proposes a second route (i.e., the displayed safer route 206f in FIG. 2F) to the user to select (or decline to stay on the current route) to avoid the locations of such other individuals.

In certain embodiments, to assist in operating as a warning system to users, the system employs geotracking of all users (or users assigned a warning code at or above a designated level) in predetermined time increments, such as five minute increments, in a secure database (that cannot be connected to the users personal information, only their application identifier as discussed above). In these embodiments, based on the warning codes associated with individuals of known locations, public health departments have the ability to map infected individuals and those they may have come into close contact with over a designated period of time, such as a 2 week period, and message those individuals to see their medical professional to be tested. For example, the system maintains, on a rolling basis, thousands of geographic data points accumulated per user (e.g., the system monitors at least 6720 geographic data points), wherein the system is operable to identify all individuals that have come within a designated distance of suspected infected individuals and provide information to such individuals regarding this potential contact.

In these embodiments, the geotracking of persons at risk of infection and the system's determination of potential infection clusters benefits both health public organizations and private organizations (i.e., airports, hotels, office buildings, live events venues, insurance companies) that want to do what is possible to reduce the spread of all infectious diseases, especially those that present a significant public health danger. For example, to combat the spread of infectious diseases, when a first employee of a businesses is infected with a contagion and/or comes in contact with someone infected with a contagion (as determined by the geotracking functionality of the system), to verify employee safety, the employer can, with permission from the first employee (whose identity remains unknown to the employer and only the unique identity code associated with that first employee is known), utilize the system to anonymously disseminate this information to other employees, such as by changing the scores and/or color codes of other employees to reflect such changes in risk (and thereby reduce the possibility that other employees infect each other and/or the general public). In another example, to combat the spread of infectious diseases, when a member of a civic group is infected with a contagion and/or comes in contact with someone infected with a contagion (as determined by the geotracking functionality of the system), to verify safety of others in the group, an administrator of the civic group can, with permission from the potentially infected member (whose identity remains unknown to the administrator of the civic group and only the unique identity code associated with that infected member is known), utilize the system to anonymously disseminate this information to other members of the civic group, such as by changing the scores and/or color codes of other member to reflect such changes in risk (and thereby reduce the possibility that other members of the group infect each other and/or the general public). It should be appreciated that in these examples, no end-user of the system will be required to present their identification to any entity (outside of potentially having their identity provided, within the confines of doctor-patient confidentially, to healthcare providers as discussed above) and thus the identification of the end-system will remain anonymous. Moreover, even if a first end-user attempts to use the mobile phone of a second end-user to improperly gain access to a venue, since the application is running in the background of the mobile phone of the second end-user, the system will identify differences in voice patterns from the first end-user to the second end-user and the system will issue a warning to the mobile phone of the second end-user regarding the potential improper use of the application being executed on another's mobile device. In this instance, the system additionally temporarily disables the mobile phone of the second end-user from certain functionality (e.g., the display of a score and/or color code indicating a relatively healthy individual) until the system determines, via voice pattern analysis, that the mobile phone is returned to the second-end user associated with the original voice pattern.

In certain embodiments, to incentive individuals to utilize the system disclosed herein, the system offers such individuals one or more rewards in association with different activities undertaken by the individuals. In one such embodiment, the system employs a point-based system wherein an individual accumulates a quantity of points for completing certain tasks associated with the system. For example, for logging into the system (via the mobile device application 202 of the mobile device 204 of FIG. 2G) and enabling healthcare (or non-healthcare) data to be anonymously collected for thirty days in a row, the system provides the individual with ten points (which may be viewed under the individual's profile page 206g of FIG. 2G). In another example, for taking an alternative route to a destination as suggested by the application, the system provides the individual with twenty points. As seen by these examples, the system awards points to one or more individuals to facilitate the tracking and tracing of individuals and/or to facilitate such individuals changing their behavior patterns. In these embodiments, in addition to accumulating points, the system enables the individuals to redeem points for one or more goods and/or services. For example, after accumulating points for avoiding, based on advice from the application, one or more venues the individual historically visited, the system enables the individual to redeem such points for a public health department funded gift card to a user selected retailer.

Accordingly, the system of the present disclosure utilizes data obtained from data gathering devices associated with an individual (e.g., wearable devices and the individual's mobile phone) and/or data provided by the individual (or their agents) in accordance with an AI algorithm (that creates and maintains a comprehensive risk assessment for all individuals) to monitor individuals objective signs and symptoms of being infected with a contagion, wherein the system generates relatively easy to understand numerical scores and color codes pertaining to a susceptibility of the individual of being infected with one or more identified contagions and/or pertaining to a probability of an individual being infected with (and correspondingly infecting others with) a contagion. In addition to conveying such information in a relatively easy to understand manner, the system enables public health departments with anonymous access to the full suite of features in the event they need to track and trace a public health issue.

In certain embodiments, the system and method of the present disclosure additionally or alternatively enables for the real time monitoring of signs and symptoms of individuals (whose identity remains anonymous to the system) suffering from chronic diseases (e.g., heart diseases, nutrition and metabolic diseases, oncologic conditions, pulmonary diseases) and/or from illnesses that require long-term and recurrent treatment. In certain embodiments, following the express approval of such individuals to permit the monitoring of various healthcare data (and, in certain instances, location data) associated with such individuals, the system operates to periodically deliver, such as hourly, daily or in real-time, the monitored healthcare data (and, in certain instances, location data) to one or more devices associated with one or more healthcare providers and/or healthcare providing facilities associated with the individual. In certain other embodiments, the system intermediates the communication of the monitored healthcare data from one or more devices associated with the individual to one or more devices associated with the healthcare provider and/or healthcare providing facilities associated with the individual by first communicating the monitored healthcare data to an artificial intelligence component of the system. The artificial intelligence component of these embodiments operates to correlate the evolution of the monitored healthcare data with alarm levels for different aspects of the healthcare data and to aggregate the healthcare data into one or more medical predictive algorithms separately developed for different diseases (which are based on the current protocols of the CDC and/or WHO). In one such embodiment, following the healthcare data being communicated to the artificial intelligence component of the system for analysis, the system operates to communicate the monitored healthcare data (and, in certain instances, location data) to one or more devices associated with one or more healthcare providers and/or healthcare providing facilities associated with the individual. In another such embodiment, following the healthcare data being communicated to the artificial intelligence component of the system for analysis, the system operates to communicate various levels of alarms (as the need arises with respect to one or more conditions of the individual) to one or more devices associated with one or more healthcare providers and/or healthcare providing facilities associated with the individual. In these embodiments, the monitored healthcare data (and/or levels of alarms) communicated to one or more devices associated with the healthcare provider and/or healthcare providing facility enables the healthcare provider to assess a disease evolution, proper treatment and eventual requirement for a consult with the individual if the need arises. It should be appreciated that all collected data, represented by signs and symptoms of any disease or condition and/or any healthcare provider recommendations are connected to the identity of the individual only for the healthcare provider, acting under applicable doctor-patient privilege, with the identity of the individual remaining anonymous to the system and non-qualified healthcare provider operators of the system.

In certain embodiments, the system of the present disclosure includes one or more computing devices housing executable software used to facilitate one or more components of the system and/or one or more components of other systems which interface with one or more components of the system. One or more instances of the computing device may be utilized to implement any, some, or all of the components of any system disclosed herein. Computing device includes a memory element. Memory element may include a computer readable medium for implementing any component of any system disclosed herein, and for implementing particular system transactions. Computing device also contains executable software, some of which may or may not be unique to the system.

In some embodiments, the system is implemented in software, as an executable program, and is executed by one or more special or general purpose digital computer(s), such as a mainframe computer, a commodity server, a personal computer (desktop, laptop or otherwise), a personal digital assistant, or other handheld or mobile computing device, such as a mobile phone. Therefore, computing device may be representative of any computer in which the system resides or partially resides.

Generally, in terms of hardware architecture, computing device includes a processor, a memory, and one or more input and/or output (I/O) devices (or peripherals) that are communicatively coupled via an interface. Interface may be one or more buses or other wired or wireless connections. Interface may have additional elements, which are omitted for simplicity, such as controllers, buffers (caches), drivers, transmitters, and receivers to facilitate external communications with other like or dissimilar computing devices. Further, interface may include address, control, and/or data connections to enable internal communications among the other computer components.

Processor is a hardware device for executing software, particularly software stored in memory. Processor can be any custom made or commercially available processor. Processor may also represent multiple parallel or distributed processors working in unison.

Memory can include any one or a combination of volatile memory elements (e.g., random access memory (RAM, such as DRAM, SRAM, SDRAM, etc.)) and nonvolatile memory elements (e.g., ROM, hard drive, flash drive, CDROM, etc.). It may incorporate electronic, magnetic, optical, and/or other types of storage media. Memory can have a distributed architecture where various components are situated remote from one another, but are still accessed by processor. These other components may reside on devices located elsewhere on a network or in a cloud arrangement.

The software in memory may include one or more separate programs. The separate programs comprise ordered listings of executable instructions for implementing logical functions. The software in memory may include the system in accordance with the present disclosure, and a suitable operating system (O/S). The operating system O/S will depend on the type of computing device. Operating system essentially controls the execution of other computer programs, such as the system, and provides scheduling, input-output control, file and data management, memory management, and communication control and related services.

Steps and/or elements, and/or portions thereof of the invention may be implemented using a source program, executable program (object code), script, or any other entity comprising a set of instructions to be performed. Furthermore, the software embodying the invention can be written as (a) an object oriented programming language, which has classes of data and methods, or (b) a procedural programming language, which has routines, subroutines, and/or functions. That is, computer program code for carrying out operations for aspects of the present disclosure may be written in any combination of one or more programming languages, including an object oriented programming language such as Java, Scala, Smalltalk, Eiffel, JADE, Emerald, C++, C#, VB.NET, Python or the like, conventional procedural programming languages, such as the “C” programming language, Visual Basic, Fortran 2003, Perl, COBOL 2002, PHP, ABAP, dynamic programming languages such as Python, Ruby and Groovy, or other programming languages. The program code may execute entirely on a local computer, partly on the local computer, as a stand-alone software package, partly on the local computer and partly on a remote computer or entirely on the remote computer or server. In the latter scenario, the remote computer may be connected to the local computer through any type of network, including a local area network (LAN) or a wide area network (WAN), or the connection may be made to an external computer (for example, through the Internet using an Internet Service Provider) or in a cloud computing environment or offered as a service such as a Software as a Service (SaaS). Components of the system may also be written in a proprietary language developed to interact with these known languages.

I/O device may include input devices such as a keyboard, a mouse, a scanner, a microphone, a touch screen, a bar code reader, or an infra-red reader. It may also include output devices such as a printer, a video display, an audio speaker or headphone port or a projector. I/O device may also comprise devices that communicate with inputs or outputs, such as a short-range transceiver (RFID, Bluetooth, etc.), a telephonic interface, a cellular communication port, a router, or other types of network communication equipment. I/O device may be internal to computing device, or may be external and connected wirelessly or via connection cable, such as through a universal serial bus port.

When computing device is in operation, processor is configured to execute software stored within memory, to communicate data to and from memory, and to generally control operations of computing device pursuant to the software. The system and operating system, in whole or in part, may be read by processor, buffered within processor, and then executed.

In the context of this document, a “computer-readable medium” may be any means that can store, communicate, propagate, or transport data objects for use by or in connection with the system. The computer readable medium may be for example, an electronic, magnetic, optical, electromagnetic, infrared, or semiconductor system, apparatus, device, propagation medium, or any other device with similar functionality. More specific examples (a non-exhaustive list) of the computer-readable medium would include the following: an electrical connection (electronic) having one or more wires, a random access memory (RAM) (electronic), a read-only memory (ROM) (electronic), an erasable programmable read-only memory (EPROM, EEPROM, or Flash memory) (electronic), an optical fiber (optical), and a portable compact disc read-only memory (CDROM) (optical). Note that the computer-readable medium could even be paper or another suitable medium upon which the program is printed, as the program can be electronically captured, via, for instance, optical scanning of the paper or other medium, then compiled, interpreted or otherwise processed in a suitable manner if necessary, and stored in a computer memory. The system can be embodied in any type of computer-readable medium for use by or in connection with an instruction execution system or apparatus, such as a computer.

For purposes of connecting to other computing devices, computing device is equipped with network communication equipment and circuitry. In certain embodiments, the network communication equipment includes a network card such as an Ethernet card, or a wireless connection card. In a preferred network environment, each of the plurality of computing devices on the network is configured to use the Internet protocol suite (TCP/IP) to communicate with one another. It will be understood, however, that a variety of network protocols could also be employed, such as IEEE 802.11 Wi-Fi, address resolution protocol ARP, spanning-tree protocol STP, or fiber-distributed data interface FDDI. It will also be understood that while one embodiment of the invention is for each computing device to have a broadband or wireless connection to the Internet (such as DSL, Cable, Wireless, T-1, T-3, OC3 or satellite, etc.), the principles of the invention are also practicable with a dialup connection through a standard modem or other connection means. Wireless network connections are also contemplated, such as wireless Ethernet, satellite, infrared, radio frequency, Bluetooth, near field communication, and cellular networks.

Any process descriptions or blocks in figures should be understood as representing modules, segments, or portions of code which include one or more executable instructions for implementing specific logical functions or steps in the process, and alternate implementations are included within the scope of the embodiments of the invention in which functions may be executed out of order from that shown or discussed, including substantially concurrently or in reverse order, depending on the functionality involved, as would be understood by those having ordinary skill in the art.

It should be emphasized that the above-described embodiments of the invention are possible examples of implementations, merely set forth for a clear understanding of the principles of the invention. Many variations and modifications may be made to the above-described embodiment(s) of the invention without substantially departing from the spirit and principles of the invention. All such modifications are intended to be included herein within the scope of this disclosure and the invention and protected by the following claims.

1. A system comprising:
a processor; and
a memory device that stores a plurality of instructions that, when executed by the processor, cause the processor to:
receive, from a wearable device, tracked healthcare data associated with an individual,
determine, based on the tracked healthcare data associated with the individual, a susceptibility of that individual of being infected with a contagion, and
cause a display device to display at least one of:
a numerical score associated with the determined susceptibility of that individual of being infected with the contagion, and
a color code associated with the determined susceptibility of that individual of being infected with the contagion.

2. The system of claim 1, wherein different numerical scores are associated with different susceptibilities of that individual of being infected with the contagion, and different color codes are associated with different susceptibilities of that individual of being infected with the contagion.

3. The system of claim 1, wherein the memory device stores a plurality of further instructions that, when executed by the processor, cause the processor to determine the susceptibility of that individual of being infected with the contagion based on the tracked healthcare data associated with the individual relative to a medical predictive algorithm.

4. The system of claim 1, wherein the memory device stores a plurality of further instructions that, when executed by the processor, cause the processor to:
receive, from the wearable device, additional tracked healthcare data associated with the individual,
determine, based on the additional tracked healthcare data associated with the individual, an updated susceptibility of that individual of being infected with the contagion, and
cause the display device to display at least one of:
an updated numerical score associated with the determined updated susceptibility of that individual of being infected with the contagion, and
an updated color code associated with the determined updated susceptibility of that individual of being infected with the contagion.

5. The system of claim 1, wherein the tracked healthcare data further comprises at least one of data received from a non-wearable device, objective data provided by the individual, subjective data provided by the individual, and data provided by a public health authority.

6. The system of claim 1, wherein the memory device stores a plurality of further instructions that, when executed by the processor, cause the processor to:
receive, from the wearable device, location data associated with the individual,
determine, based on the tracked healthcare data associated with the individual and the location data associated with the individual, the susceptibility of that individual of being infected with the contagion.

7. The system of claim 1, wherein the individual is associated with an anonymous identity code.

8. The system of claim 1, wherein the display device comprises at least one of: a display device of the wearable device, a display device of a non-wearable device associated with the individual, a display device associated with a healthcare provider associated with the individual, and a display device associated with a public health system authority.

9. The system of claim 1, wherein the memory device stores a plurality of further instructions that, when executed by the processor, cause the processor to cause the display device to display an image of the individual.

10. A system comprising:
a processor; and
a memory device that stores a plurality of instructions that, when executed by the processor, cause the processor to:
for each of a plurality of anonymous individuals, receive:
tracked healthcare data associated with that anonymous individual, and
location data associated with that anonymous individual,
for each of the plurality of anonymous individuals, determine a numerical score associated with a susceptibility of that anonymous individual of being infected with a contagion,
determine a potential infection cluster based on the location data associated with the anonymous individuals and the determined numerical scores, and
cause a display device to display data associated with the determined potential infection cluster.

11. The system of claim 10, wherein the data associated with the determined potential infection cluster comprises instructions to avoid a location associated with the determined potential infection cluster.

12. A method of operating a system, the method comprising:
receiving, from a wearable device, tracked healthcare data associated with an individual,
determining, by a processor and based on the tracked healthcare data associated with the individual, a susceptibility of that individual of being infected with a contagion, and
displaying, by a display device, at least one of:
a numerical score associated with the determined susceptibility of that individual of being infected with the contagion, and
a color code associated with the determined susceptibility of that individual of being infected with the contagion.

13. The method of claim 12, wherein different numerical scores are associated with different susceptibilities of that individual of being infected with the contagion, and different color codes are associated with different susceptibilities of that individual of being infected with the contagion.

14. The method of claim 12, further comprising determining, by the processor, the susceptibility of that individual of being infected with the contagion based on the tracked healthcare data associated with the individual relative to a medical predictive algorithm.

15. The method of claim 12, further comprising:
receiving, from the wearable device, additional tracked healthcare data associated with the individual,
determine, by the processor and based on the additional tracked healthcare data associated with the individual, an updated susceptibility of that individual of being infected with the contagion, and
displaying, by the display device, at least one of:
an updated numerical score associated with the determined updated susceptibility of that individual of being infected with the contagion, and
an updated color code associated with the determined updated susceptibility of that individual of being infected with the contagion.

16. The method of claim 12, wherein the tracked healthcare data further comprises at least one of data received from a non-wearable device, objective data provided by the individual, subjective data provided by the individual, and data provided by a public health authority.

17. The method of claim 12, further comprising:
receiving, from the wearable device, location data associated with the individual,
determining, by the processor and based on the tracked healthcare data associated with the individual and the location data associated with the individual, the susceptibility of that individual of being infected with the contagion.

18. The method of claim 12, wherein the individual is associated with an anonymous identity code.

19. The method of claim 12, wherein the display device comprises at least one of: a display device of the wearable device, a display device of a non-wearable device associated with the individual, a display device associated with a healthcare provider associated with the individual, and a display device associated with a public health system authority.

20. The method of claim 12, further comprising displaying, by the display device, an image of the individual.
